(19) United States
(12) Patent Application Publication
(10) Pub. No.: US 2004/0259898 A1
(43) Pub. Date: Dec. 23, 2004

(54) Use of methylnaltrexone in treating gastrointestinal dysfunction in equines

(76) Inventor: Jonathan Moss, Chicago, IL (US)

Correspondence Address: ROTHWELL, FIGG, ERNST & MANBECK, P.C., 1425 K STREET, N.W., SUITE 800, WASHINGTON, DC, 20005, US

(21) Appl. No.: 10/493,568

(22) PCT Filed: Oct. 28, 2002
(86) PCT No.: PCT/US02/34458

Related U.S. Application Data
(60) Provisional application No. 60/354,278, filed on Feb. 4, 2002.

(51) Int. Cl. A61K 31/485

(52) U.S. Cl. 514/282

(57) ABSTRACT

Systems and methods are described for using methylnaltrexone in treating inhibition of gastrointestinal motility in equines. A method for preventing and treating opioid-induced and non-opioid-induced gastrointestinal dysfunction includes administering a quaternary derivative of noroxymorphone to an equine before or after the onset of the gastrointestinal dysfunction.

### CROSS-REFERENCE TO RELATED APPLICATION

This application is related to and claims a benefit of priority under 35 U.S.C. 119(e) from, copending U.S. Ser. No. 60/354,278, filed Feb. 4, 2002, the entire contents of which are hereby expressly incorporated by reference for all purposes.

### BACKGROUND OF THE INVENTION

1. Field of the Invention

The invention relates generally to the field of equine medicine. More particularly, the invention relates to inhibition of equine gastrointestinal motility. Specifically, a preferred implementation of the invention relates to the treatment of inhibition of equine gastrointestinal motility.

2. Discussion of the Related Art

The inventory of equines in the United States as of Jan. 1, 1999 totaled 5.32 million head, up 1.3 percent from the 5.25 million head on Jan. 1, 1998. Inventory at the start of 2002 is just shy of 5.5 million head. Alternative reports suggest as many as 6.9 million horses in North America. Equine includes horses, ponies, mules, burros, and donkeys. Texas ranked first in equine inventory with 600,000 head followed by California, and Tennessee with 240,000 and 190,000 head, respectively. Florida, Oklahoma, and Pennsylvania tied for fourth with an inventory of 170,000 head. Ohio ranked seventh with 160,000 head, followed by Kentucky, Minnesota, New York, and Washington with 155,000 head. An additional fifteen states had equine inventories of 100,000 head or more.

Equine located on farms totals approximately 60% while non-farm animals accounted for 39.1 percent of the total. Non-farm horses are used for recreation (>40%), showing (<30%), racing (˜10%) and other purposes such as hunting (˜18%).

The total economic impact due to the U.S. horse industry approaches $112 billion. More than 7 million Americans are involved in the horse industry, including approximately 2 million owners of horses. This industry supports more than a million jobs and pays into federal, state and local governments almost $2 billion in taxes. Value of sales from equine sold in 1998 was $1.75 billion, up 6.9 percent from of $1.64 billion in 1997. The top ten states for equine sales were Kentucky, Florida, Texas, California, Virginia, New Jersey, Tennessee, New York, Pennsylvania, and Maryland.

Horses are highly susceptible to gastrointestinal distress, in particular, gastrointestinal (GI) hypoperistalsis. GI hypoperistalsis may occur in several forms in equines as well as other animals, the most notable of these forms include colic and post-operative ileus. Post-operative ileus is a widely known phenomenon, oftentimes appearing on a vet's post-operative checklist for vital signs as a colic scale, alongside checkpoints for pulse and blood pressure.

The inhibition of equine gastrointestinal motility, such as colic and constipation, may be fatal to a horse. The pain suffered by the horse who has colic is enough to send the animal into a death-inducing shock, while a long-term case of constipation may also cause the horse's death.

The main causes of colic are intestinal distension and reduced blood supply to the intestinal tract. Peristalsis of the intestine is reduced and distention will occur due to reduced movement and absorption of water and nutrients. The pressure that results from this lack of passage of material through the digestive system results in a reflex action, which causes adjoining areas to contract in spasm. Distension and reduced blood flow may be due to an accumulation of gas fluid or feed, digestive disturbances, intestinal obstructions, internal parasites, or twisted intestine (torsion and volvulus). A horse constantly swallowing air or “wind sucking” may cause chronic distension.

The primary cause of the abdominal pain is this distention. Pain is also produced when the peritoneum is stretched during attacks of colic. The first response the body makes to distension is to increase the secretion of digestive juices, which increases the pressure, and causes dehydration and imbalance in the chemical systems of the body. This can often become a feedback reaction leading to shock, which must be treated as a separate syndrome, since it is frequently the cause of colic deaths. The paralysis of the intestine also allows toxic material to escape through the stretched walls and enter the abdominal cavity, where the horse can be poisoned by his own intestinal contents.

Veterinarians often perform a rectal exam; intestinal contents and their position can indicate to the veterinarian presence or absence of intestinal motility and the location of the obstruction or impact. A stomach tube may be used to collect stomach contents or gas to help the veterinarian decide the type of disorder and the severity of the condition. Other symptom the vet will note include pulse (rate should be less than 80 per minute for a favorable prognosis), temperature, presence or absence of intestinal sounds. Generally, the prognosis is excellent when pain is due to excessive activity of the intestines, good for pain due to impaction, and very poor for pain caused by twisting or intussusception of the intestines (unless surgery is immediate).

Current treatments for horse colic are not effective. These include the use of a stomach tube to relieve gas pressure on the horse's stomach and giving antacid-antigas type medications (e.g., Maalox). Mineral oil may be administered via stomach tube to loosen the blockage. However, side effects of the use of mineral oil are depletion of stored vitamins and the blockage of vitamin absorption in the horse's stomach. Surgery is the final treatment in cases of severe colic. The risks and expense inherent in large animal surgeries makes this a treatment reserved for commercially important animals and only a few individual owners. When treating horses for opioid-related conditions, such as post-operative ileus, the medications used to treat the constipation resulting from opioid medication reduces the painkilling effects of the medication, which could result in shock and the horse's death.

Heretofore, the needs for an agent to treat or prevent opioid-induced side effects and to treat non-opioid related gastrointestinal motility problems have not been fully met. What is needed is a solution that addresses all of these requirements.

### SUMMARY OF THE INVENTION

According to an aspect of the invention, there is provided a method for treating opioid induced gastrointestinal dysfunction comprising administering a quaternary derivative of noroxymorphone to an equine after the onset of the gastrointestinal dysfunction. According to another aspect of the invention, a method for treating opioid induced gastrointestinal dysfunction comprising administering a quaternary derivative of noroxymorphone to an equine before the onset of the gastrointestinal dysfunction is provided. The quaternary derivative is methylnaltrexone, which can be administered by intravenous, intramuscular, transmucosal, transdermal, subcutaneous, epidural, spinal, peritoneal, or oral administration.

According to yet another aspect of the invention, a method is provided for treating non-opioid induced gastrointestinal dysfuntion comprising administering a quaternary derivative of noroxymorphone to an equine after the onset of the gastrointestinal dysfunction. According to another aspect of the invention, a method for treating non-opioid induced gastrointestinal dysfunction comprising administering a quaternary derivative of noroxymorphone to an equine before the onset of the gastrointestinal dysfunction is provided. The quaternary derivative is methylnaltrexone, which can be administered by intravenous, intramuscular, transmucosal, transdermal, subcutaneous, epidural, spinal, peritoneal, or oral administration.

The methylnaltrexone can be formulated with saline for administration by intravenous or intramuscular administration, or with a pharmacologically acceptable carrier, and can be administered at a dosage of 0.05 to 40.0 mg of active drug per kg body weight. The methylnaltrexone can also be an enterically coated methylnaltrexone that is administered at a dosage of 0.05 to 40.0 mg of active drug per kg body weight. The enterically coated methylnaltrexone can also be administered orally at a dosage of about 0.1 to about 10 mg/kg body weight as an enterically coated tablet or capsule, or as enterically coated granules, where the enteric coating provides time release of the methylnaltrexone.

The gastrointestinal dysfunction treated by the methylnaltrexone can be constipation, colic, post-operation ileus, or grass sickness.

These, and other, embodiments of the invention will be better appreciated and understood when considered in conjunction with the following description and the accompanying drawings.

### BRIEF DESCRIPTION OF THE DRAWINGS

The drawings accompanying and forming part of this specification are included to depict certain aspects of the invention. A clearer conception of the invention, and of the components and operation of systems provided with the invention, will become more readily apparent by referring to the exemplary, and therefore nonlimiting, embodiments illustrated in the drawings, wherein like reference numerals (if they occur in more than one view) designate the same elements. The invention may be better understood by reference to one or more of these drawings in combination with the description presented herein. It should be noted that the features illustrated in the drawings are not necessarily drawn to scale.

FIG. 1 is a graph showing mean plasma β-endorphin levels (pmol ml⁻¹±SEM) before, during, and after application of an upper lip twitch stimulus in six horses.

FIG. 2 shows the chemical structure of methylnaltrexone (MNTX).

### DETAILED DESCRIPTION

The invention and the various features and advantageous details thereof are explained more fully with reference to the nonlimiting embodiments that are illustrated in the accompanying drawings and detailed in the following description. Various substitutions, modifications, additions and/or rearrangements within the spirit and/or scope of the underlying inventive concept will become apparent to those skilled in the art from this disclosure.

I. Colic and Other Gastrointestinal Dysfunctions

Some form of colic affects approximately 10% of horses every year. The main causes of colic are intestinal distension and reduced blood supply to the intestinal tract. Peristalsis of the intestine is reduced and distention will occur due to reduced movement and absorption of water and nutrients. The pressure that results from this lack of passage of material through the digestive system results in a reflex action, which causes adjoining areas to contract in spasm. Distension and reduced blood flow may be due to an accumulation of gas fluid or feed, digestive disturbances, intestinal obstructions, internal parasites, or twisted intestine (torsion and volvulus). A horse constantly swallowing air or “wind sucking” may cause chronic distension that may lead to colic.

The primary cause of the abdominal pain is this distention. Pain is also produced when the peritoneum is stretched during attacks of colic. The first response the body makes to distension is to increase the secretion of digestive juices, which increases the pressure, and causes dehydration and imbalance in the chemical systems of the body. This can often become a feedback reaction which can lead to shock, which must be treated as a separate syndrome by the vet, since it is frequently the cause of colic deaths. The paralysis of the intestine also allows toxic material to escape through the stretched walls and enter the abdominal cavity, where the horse can be poisoned by his own intestinal contents.

There are various causes of colic and since the prognosis and treatment varies greatly with each. Early recognition and accurate determination of what type of colic the horse is experiencing is very important.

This disclosure identifies a novel approach to treating colic and other gastrointestinal motility problems in animals using methylnaltrexone (MTNX). In one embodiment of the invention, this method is used in treating equine colic, a disorder that affects approximately 10% of horses annually. It also has alternate applications for treating grass sickness, post-operative ileus and laminitis in horses. MTNX is a peripheral opiate antagonist under development for human health applications by Progenics (Tarrytown, N.Y.).

II. μ-Receptors and β-Endorphins

μ-receptors are responsible for analgesia, and for the classical or morphine-like side effects of opioids. Only a small percentage of these receptors need to be occupied in order to produce analgesia. μ-receptors are clustered in the cerebral cortex, some regions of the thalamus, and in the periaqueductal grey region of the spinal cord. They are also found in large amounts in the gut.

Some experts believe that μ-receptors should be divided into two sub-groups. μ1 receptors have a high affinity for opioids, and are associated with analgesia. μ2 receptors have a low affinity for opioids and are associated with respiratory depression and probably, in the development of physical dependence.

Therefore, MNTX is able to counteract the negative gastrointestinal effects of opioids while not decreasing the pain-reducing effects of the opioids. This is especially important when applied to equines.

Another characteristic of morphine in relation to equines, and possibly other animals, is that morphine can send in the horse into sudden rage. Conventional treatments with anti-opioid compounds have been unsuccessful, possibly due to central μ-receptors. MNTX has been shown to be minimize the severity of the morphine-induced rage in an animal.

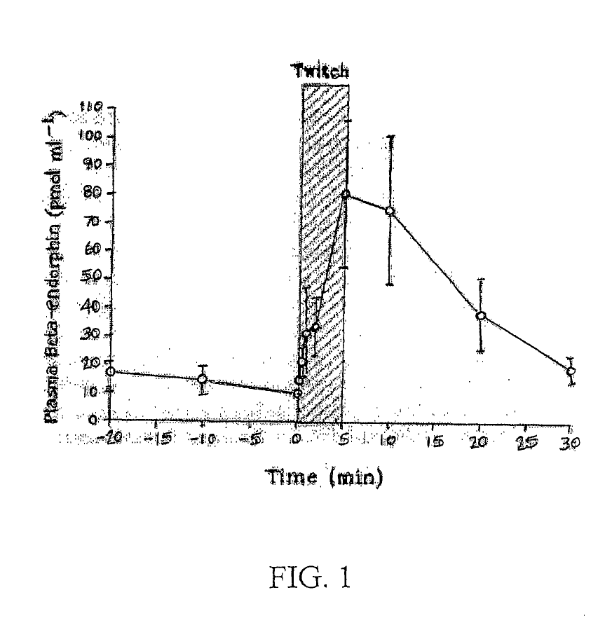

It has been shown in horses that the amount of immunoreactive β-endorphin concentration (ir β-EP) in their plasma rises dramatically when the horse is exposed to pain, such as severe abdominal pain stemming from conditions such as colic, fright, and surgical procedures. In one study, a lip twitch was applied to the muzzles of six horses for 5 minutes, and their β-EP levels were measured during the 5 minutes and for 30 minutes after the twitch was removed. The results from this study is shown in FIG. 1.

β-EP is an endogenous opioid released primarily from the adenohypophysis after post-translational differential processing of pro-opiomelanocortin (POMC). β-EP is known to be hypotensive, possibly by acting on a serotonergic pathway, and thus possibly contributing to shock. High levels of plasma β-endorphin (β-EP) levels have been associated with cardiogenic shock and endotoxemia.

Any increase in pain and stress can elevate plasma concentrations of β-EP. It has been shown that prolonged air transportation of the horse can result in sustained elevation of plasma concentrations of ir β-EP. A surgical procedure on as localized an area as a horse's eye is also enough to elevate ir β-EP levels to extremely high levels that may prove dangerous to the horse. Horses suffering from colic showed marked elevations in plasma concentrations of ir β-EP, which may have contributed to death-causing shock.

Therefore, for conditions such as post-operative ileus, the administration of MNTX can aid in decreasing the onset of shock due to elevated concentrations of β-EP by becoming attached to the μ-receptors that β-EP would normally be attached to. By inhibiting β-EP, the risk of β-EP induce shock may be minimized.

III. Methylnaltrexone

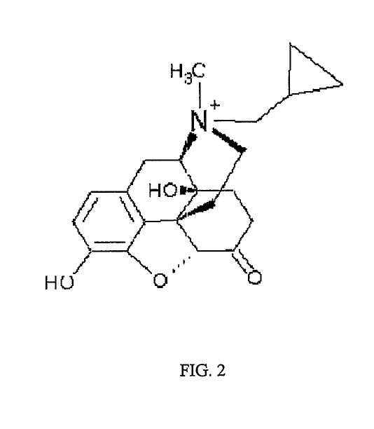

Methylnaltrexone is a quaternary amine derivative of naltrexone and a quaternary derivative of noroxymorphone, the structure of which is shown in FIG. 2. Methylnaltrexone has been found to have only 2 to 4% of the opiate antagonistic activity of naltrexone in vivo due to its inability to pass the blood-brain-barrier and bind to the opiate receptors in the central nervous system.

MNTX has been proven for use in humans in either the enterically coated form or in order to prevent or treat opioid induced side effects including dysphoria, pruritus, and urinary retention and non-opioid induced changes in gastrointestinal motility in patients. MNTX does not cross the blood-brain-barrier, and does not interfere for brain-centered relief nor does it irritate the horse to the point of risking injury to itself or its handlers.

MNTX is a specific peripheral opioid antagonist. It acts by binding to opioid receptors without activating them, thus competing with the binding of opioid drugs. MNTX targets μ-receptors, the same receptors that are targeted by opioids. MNTX is designed to block opioid side effects in the peripheral tissues of the body without interfering with ability of opioids to relieve pain via the central nervous system.

When used as a treatment for opioid- and nonopioid-induced side effects, orally administered, particularly if enteric coated, methylnaltrexone (MNTX) or other quaternary derivatives of noroxymorphone (QDMN) provides prolonged relief of the side effects. Furthermore, for treatment or prevention of delayed gastric emptying from enteric feeding and constipation, whether caused by extrinsic or endogenous opioids, enteric coating surprisingly allows for equal or better efficacy despite lower plasma levels. Idiopathic constipation, i.e., constipation that is due to causes other than exogenous administration of opioids, may be mediated by opioid sensitive mechanisms. Endogenous opioid receptors have been identified in the gut, and these receptors may modulate gut motility. Thus, administration of an opioid antagonist with peripheral action, such a methylnaltrexone or other quaternary derivatives of noroxymorphone, would block the effects of endogenous opioids.

MNTX can gain access to opioid receptors located in the gastrointestinal tract via both direct luminal access and through the plasma, thus preventing opioids from binding to these receptors and affecting gastrointestinal function.

MNTX does not, however, attach to μ receptors in the brain, however, because it was designed to inhibit its ability to cross the blood-brain barrier by lowering its lipid solubility as compared to naltrexone. This is made possible by the formation of quaternary nitrogen wherein an additional methyl group is attached to the naltrexone molecule. This confers a net positive change on the molecule and limits its ability to diffuse freely through the blood-brain barrier.

A. Enterically-Coated MNTX

In one embodiment for the prevention and/or treatment of constipation and inhibition of gastrointestinal motility, the QDNM or MNTX is enterically coated and administered orally. For oral administration, the QDNM or MNTX is formulated with pharmacologically acceptable binders to make a tablet or capsule with an enteric coating. An enteric coating is one which remains intact during passage through the stomach, but dissolves and releases the contents of the tablet or capsule once it reaches the small intestine. Most currently used enteric coatings are those which will not dissolve in low pH environments, but readily ionize when the pH rises to about 4 or 5, for example synthetic polymers such as polyacids having a pKₐ of 3 to 5.

The enteric coating may be made of any suitable composition. Preferred enteric coating compositions include alkyl and hydroxyalkyl celluloses and their aliphatic esters, e.g., methylcellulose, ethylcellulose, hydroxyethylcellulose, hydroxypropylcellulose, hydroxybutylcellulose, hydroxyethylethylcellulose, hydroxyprophymethylcellulose, hydroxybutylmethylcellulose, hydroxypropylcellulose phthalate, hydroxypropylmethylcellulose phthalate and hydroxypropylmethylcellulose acetate succinate; carboxyalkylcelluloses and their salts, e.g., carboxymethylethylcellulose; cellulose acetate phthalate; cellulose acetate trimellitate, polycarboxymethylene and its salts and derivatives; polyvinyl alcohol and its esters: polyvinyl acetate phthalate; polycarboxymethylene copolymer with sodium formaldehyde carboxylate; acrylic polymers and copolymers, e.g., methacrylic acid-methyl methacrylic acid copolymer and methacrylic acid-methyl acrylate copolymer; edible oils such as peanut oil, palm oil, olive oil and hydrogenated vegetable oils; polyvinylpyrrolidone; polyethylene glycol and its esters; natural products such as shellac, and zein.

Other preferred enteric coatings include polyvinylacetate esters, e.g., polyvinyl acetate phthalate; alkyleneglycolether esters of copolymers such as partial ethylene glycol monomethylether ester of ethylacrylate-maleic anhydride copolymer or diethyleneglycol monomethylether ester of methylacrylate-maleic anhydride copolymer, N-butylacrylate-maleic anhydride copolymer, isobutylacrylate-maleic anhydride copolymer or ethylacrylate-maleic anhydride copolymer; and polypeptides resistant to degradation in the gastric environment, e.g., polyarginine and polylysine. Other suitable coatings and methods to make and use such formulations are well known to those skilled in the art.

Mixtures of two or more of the above compounds may be used as desired. The presently preferred enteric coating comprises cellulose acetate phthalate.

The enteric coating material may be mixed with various excipients including plasticizers such as triethyl citrate, acetyl triethyl citrate, diethyl phthalate, dibutyl phthalate, dibutyl subacute, dibutyl tartrate, dibutyl maleate, dibutyl succinate and diethyl succinate and inert fillers such as chalk or pigments.

The composition and thickness of the enteric coating may be selected to dissolve immediately upon contact with the digestive juice of the intestine. Alternatively, the composition and thickness of the external coating may be selected to be a time-release coating which dissolves over a selected period of time, as is well known in the art.

The amount of enteric coating depends on the particular enteric coating composition used and is preferably sufficient to substantially prevent the absorption of MNTX in the stomach.

Hydroxyalkyl celluloses and their aliphatic esters, carboxyalkyl celluloses and their salts, polycarboxymethylene and its salts and derivatives, polyvinyl alcohol and its esters, polycarboxymethylene copolymer with sodium formaldehyde carboxylates, polyvinylpyrrolidone, and polyethylene glycol and its esters can be applied as enteric coatings by first dissolving the compound in a minimum amount of water. Alcohol is then added to the point of incipient cloudiness. The mixture can then be applied by conventional techniques.

Application of cellulose acetate phthalate may be accomplished by simply dissolving the cellulose acetate phthalate in a minimum amount of alcohol and then applying by conventional techniques. Hydrogenated vegetable oils may be applied by first dissolving the oil in a minimal amount of a non-polymer solvent, such as methylene chloride, chloroform or carbon tetrachloride, then adding alcohol to the point of incipient cloudiness and then applying by conventional techniques.

In one embodiment, the MNTX is coated with Eudragit L100 or S100, a methacrylic acid copolymer enteric coating, at a 50% coating level to provide stability at gastric pH and dissolution at gut pH per a US Pharmacopeia (USP) standard for enteric coatings.

B. MNTX Administration

MNTX has been shown to be effective in preventing and treating opioid-induced constipation and changes in equine gut motility via the oral administration of an enteric coated quaternary derivatives of noroxymorphone (QDNM), particularly methylnaltrexone (MNTX). Administration of non-enterically coated MNTX results in rapid absorption of MNTX through the equine stomach and early peak and sustained high levels of MNTX in the plasma. However, an enteric coating on the QDNM, designed to prevent dissolution and subsequent absorption of the drug in the stomach, would be expected to produce delayed elevation of plasma levels of the QDNM, and to produce a lower peak plasma level. Suprisingly, however, administration of enterically coated MNTX has been found to result in substantially lower plasma levels as compared to non-enterically coated MNTX at the same dosage level, and surprisingly and unexpectedly resulted in enhanced efficacy in the reversal of opioid-induced decreases in equine gastrointestinal motility. In fact, it has been found that as compared to non-enterically coated MNTX, a significantly lower dose, e.g., less than half the amount of coated MNTX can be used if enterically -coated to achieve the same levels of relief of opioid-induced constipation. Moreover, such reduced dosage levels of MNTX administered with an enteric coating results in exceedingly low peak and sustained plasma levels of MNTX, greatly reducing the potential adverse side effects of the MNTX. This improvement in the clinical indication for use of MNTX has led to an increased therapeutic index for this drug.

When used as a treatment for the opioid- and nonopioid-induced side effects of constipation and reduction of equine gastrointestinal motility, orally administered, particularly if enterically coated, MNTX or other quaternary derivatives of noroxymorphone provide prolonged relief of the side effects. MNTX has been demonstrated to have the ability to block the equine gastrointestinal effects of opioids on motility when administered intravenously or orally.

Furthermore, for treatment or prevention of equine constipation and delayed gastrointestinal emptying, whether caused by extrinsic or endogenous opioids, enteric coating surprisingly allows for equal or better efficacy despite lower plasma levels. Idiopathic constipation, i.e., constipation that is due to causes other than exogenous administration of opioids, may be mediated by opioid sensitive mechanisms. Endogenous opioid receptors have been identified in the gut, and these receptors may modulate gut motility. Thus, administration of an opioid antagonist with peripheral action, such a methylnaltrexone or other quaternary derivatives of noroxymorphone, would block the effects of endogenous opioids. Quaternary derivatives of noroxymorphone are described in full in U.S. Pat. No. 4,176,186.

Opioids are typically administered at a morphine equivalent dosage of: 0.005 to 0.15 mg/kg body weight for intrathecal administration; 0.05 to 1.0 mg/kg body weight for intravenous administration; 0.05 to 1.0 mg/kg body weight for intramuscular administration; 0.05 to 1.0 mg/kg body weight/hour for transmucosal or transdermal administration. “Morphine equivalent dosage” is meant to be representative doses of other opioids which equal one milligram of morphine, for example 10 mg meperidine, 1 mg methadone, and 80 μg fentanyl.

In accordance with the present invention, methylnaltrexone is administered at a dosage of: 0.1 to 40.0 mg/kg body weight for equine oral administration, including enteric coated methylnaltrexone.

The administration of the methylnaltrexone is preferably commenced prior to administration of the opioid to prevent opioid-induced inhibition of gastrointestinal motility or constipation. It is desirable to commence internal administration of methylnaltrexone about 20 minutes prior to administration of opioids in order to prevent these opioid-induced side effects. While the prevention of symptoms is preferred, methylnaltrexone administration may also be commenced after the administration of the opioid or after the onset of opioid induced symptoms as a treatment for those symptoms.

Methylnaltrexone is rapidly absorbed after oral administration from the stomach and bowel. Initial plasma levels of the drug are seen within 5-10 minutes of the administration of non-enteric coated compound. Addition of an enteric coating which prevents gastric absorption is associated with lower plasma levels of the methylnaltrexone. Surprisingly, the addition of an enteric coating (i.e., a coating which will prevent degradation or release in the stomach, but will release drug in the small and large bowel) enhances the efficacy of methylnaltrexone in the prevention of decreases in gut motility by intravenously administered opioids such as morphine.

For intravenous or parenteral administration, methylnaltrexone is formulated with saline or other physiologically acceptable carriers; for intramuscular administration, the methylnaltrexone is formulated with saline or other pharmacologically acceptable carriers; for transmucosal administration the methylnaltrexone is formulated with a sugar and cellulose mix or other pharmacologically acceptable carriers known in the art; and for oral administration, the methylnaltrexone may be formulated with pharmacologically acceptable binders to make a tablet or capsule with or without an enteric coating. Methods for such formulations are well known to those skilled in the art.

Other methods of administrating MNTX that would use a formulation similar to that of intravenous administration include epidural, spinal, catheter, peritoneal, and subcutaneous administration.

For transdermal administration, any art-known transdermal application may be used, including using a patch applied to the skin with a membrane of sufficient permeability to allow diffusion of MNTX at a fixed rate in the range of 1.0 to 10.0 mg/hr. The rate of administration may be varied by varying the size of the membrane contact area and/or applying an electrical wiring potential to a drug reservoir. The patch preferably holds 25 mg to 1 gram of available drug in the reservoir plus additional drug as needed for the mechanics of the system.

In the above description, methylnaltrexone is used as an example of a particularly effective QDNM. It is apparent that other QDNM's may be used as desired. MNTX may also be administered in combination with certain opioids as an analgesia

Based on its properties, MNTX is suitable for situations such as the ones listed above. The administering of MNTX in conjunction with opioids would alleviate the pain while preventing constipation and reducing the levels of β-endorphins in the plasma.

### REFERENCES

All of the references listed herein are incorporated by reference in their entirety.

U.S. Pat. No. 4,176,186

U.S. Pat. No. 4,311,833

U.S. Pat. No. 4,377,568

U.S. Pat. No. 4,385,078

U.S. Pat. No. 4,457,907

U.S. Pat. No. 4,462,839

U.S. Pat. No. 4,518,433

U.S. Pat. No. 4,556,552

U.S. Pat. No. 4,606,909

U.S. Pat. No. 4,615,885

U.S. Pat. No. 4,670,287

U.S. Pat. No. 4,719,215

U.S. Pat. No. 4,861,781

U.S. Pat. No. 5,102,887

U.S. Pat. No. 5,536,507

U.S. Pat. No. 5,567,423

U.S. Pat. No. 5,591,433

U.S. Pat. No. 5,597,564

U.S. Pat. No. 5,609,871

U.S. Pat. No. 5,614,222

U.S. Pat. No. 5,626,875

U.S. Pat. No. 5,629,001

U.S. Pat. No. 5,972,954

U.S. Pat. No. 6,274,591

Douglas, Janet, “Colic”, http://www.erc.on.ca/colic2.htm.

Gustavson, Carrie, “Colic Means Pain in the Gut”, Univ. of Illinois Urbana-Champagne, College of Veterinary Medicine, Continuing Education, Pet Column for the week of Aug. 21, 2000. http://www.cvm.uiuc.edu/petcolumns/showarticle.cfm?id=12.

McCarthy, R. N., Jeffcott, L. B., Clarke, I. J., “Preliminary Studies on the Use of Plasma β-Endorphin in Horses as an Indicator of Stress and Pain”, Journal of Equine Veterinary Science, Vol. 13, No. 4, pp. 216-219, 1993.

Progenics Pharmaceuticals, Inc., Tarrytown, N.Y., http://www.progenics.com.

Proudman, C. J., “Intestinal Motility and Impactions”, Equine Vet. J, Suppl. Vol. 32, pp. 8-10, 2000

Remington, The Science and Practice of Pharmacy, 19th ed. (1995) Mack Publishing Company, Easton, Pa.

1. A method for treating gastrointestinal dysfunction in an engine induced by elevated concentrations of endoqenous opioids, while maintaining the pain-reducing effects of the opioids, comprising administering an effective amount of a quaternary derivative of noroxymorphone to the equine after the onset of the gastrointestinal dysfunction, thereby treating the gastrointestinal dysfunction without precipitating pain in the equine.

2. The method of claim 1, wherein the quaternary derivative is methylnaltrexone.

3. The method of claim 2, wherein the methylnaltrexone is administered by a method selected from the group consisting of intravenous, intramuscular, transmucosal, transdermal, subcutaneous, epidural, spinal, peritoneal, and oral administration.

4. The method of claim 2, wherein the methylnaltrexone is administered by intravenous, intramuscular, or subcutaneous administration.

5. The method of claim 2, wherein the methylnaltrexone is formulated with a pharmacologically acceptable carrier.

6. The method of claim 2, wherein the methylnaltrexone is administered at a dosage of 0.05 to 40.0 mg of active drug per kg body weight.

7. (Canceled).

8. (Canceled).

9. (Canceled).

10. (Canceled).

11. (Canceled).

12. The method of claim 1, wherein the gastrointestinal dysfunction is constipation.

13. The method of claim 1, wherein the gastrointestinal dysfunction is colic.

14. The method of claim 1, wherein the gastrointestinal dysfunction is post-operation ileus

15. The method of claim 1, wherein the gastrointestinal dysfunction is grass sickness.

16. A method for preventing gastrointestinal dysfunction in an equine induced by elevated concentrations of endogenous opioids, while maintaining the pain-reducing effects of the opioids, comprising administering an effective amount of a quaternary derivative of noroxymorphone to the equine before the onset of the gastrointestinal dysfunction, thereby preventing the gastrointestinal dysfunction.

17. The method of claim 16, wherein the quaternary derivative is methylnaltrexone.

18. The method of claim 17 wherein the methylnaltrexone is administered by a method selected from the group consisting of intravenous, intramuscular, transmucosal, transdermal, subcutaneous, epidural, spinal, peritoneal and oral administration.

19. The method of claim 17, wherein the methylnaltrexone is administered by intravenous, intramuscular, or subcutaneous administration.

20. The method of claim 17, wherein the methylnaltrexone is formulated with a pharmacologically acceptable carrier.

21. The method of claim 17, wherein the methylnaltrexone is administered at a dosage of 0.05 to 40.0 mg of active drug per kg body weight.

22. (Canceled).

23. (Canceled).

24. (Canceled).

25. (Canceled).

26. (Canceled)

27. The method of claim 16, wherein the gastrointestinal dysfunction is constipation.

28. The method of claim 16, wherein the gastrointestinal dysfunction is colic.

29. The method of claim 16, wherein the gastrointestinal dysfunction is post-operation ileus

30. The method of claim 16, wherein the gastrointestinal dysfunction is grass sickness.

31-60. (Canceled).
